(19) United States
(12) Patent Application Publication
(10) Pub. No.: US 2006/0105382 A1
(43) Pub. Date: May 18, 2006

(54) Microfluidic channels with attached biomolecules

(76) Inventors: Yingjie Liu, Chandler, AZ (US); Piotr Grodzinski, Chandler, AZ (US); Cory Rauch, Gilbert, AZ (US); Thomas J. Smekal, Phoenix, AZ (US)

Correspondence Address: MOTOROLA, INC., 1303 EAST ALGONQUIN ROAD, IL01/3RD, SCHAUMBURG, IL, 60196, US

(21) Appl. No.: 11/320,101

(22) Filed: Dec. 28, 2005

Related U.S. Application Data
(62) Division of application No. 10/242,872, filed on Sep. 13, 2002.

(51) Int. Cl. C12Q 1/68; G01N 33/53; C12M 1/34; H01L 21/00

(52) U.S. Cl. 435/6; 435/7.1; 435/287.2; 438/1

(57) ABSTRACT

An exemplary system and method for bonding substrate layers in the presence of chemically active species to form functionalized microfluidic surfaces is disclosed as comprising inter alia a first substrate (100), a second substrate (200), a chemically functional species (120) attached to first substrate (100), and a radiatively absorptive mask material (130) disposed substantially between first substrate (100) and second substrate (200). Mask material (130) is suitably adapted to effectively bond first substrate (100) with second substrate (200) upon exposure of the composite structure to radiation of a predetermined, user-selectable wavelength. Disclosed features and specifications may be variously controlled, adapted or otherwise optionally modified to improve certain device fabrication parameters and/or performance metrics.

### FIELD OF INVENTION

The present invention relates to microfluidics, and more particularly, in one representative and exemplary embodiment, to a system and method for fabricating micro-channels with attached biological molecules.

### BACKGROUND

Microfluidic systems and environments have received much interest in chemical and biological research as well as in a variety of diagnostics applications. Representative advantages of microfluidic environments include significant reduction in reagent volumes, sample input volumes, power, form-factors and assay development times. Additionally, the low cost and stability of various plastic materials generally renders polymer substrates well-suited for the manufacturing of microfluidic devices in bulk quantities. Accordingly, substantial effort has been brought to bear on the development of manufacturing techniques for the use of polymer materials on a micro-scale.

One of the goals of fabrication of such devices is to create robust bonds between polymer layers. Conventional processes for the bonding of polymer surfaces include friction induced thermal bonding, solvent bonding, thermal compression bonding and adhesive bonding. These methods, however, are not generally adaptable for certain microfluidic applications involving, for example, the disposition of biological or other chemical species within or on the surfaces of micro-channels themselves.

In order to localize molecules to a specific chamber or channel within a microfluidic device, the molecules must generally be deposited during manufacturing at some time prior to sealing of the device. The disposition of active material typically involves attachment of active species such as DNA, RNA, antibodies, antigens, enzymes, bio-organic molecules, and/or the like to the interior channel or chamber walls prior to sealing of the microfluidic device. Once attached, these materials are generally sensitive inter alia to heat, physical agitation and the presence or absence of solvents. As a result, conventional bonding methods will often be destructive to sensitive biologically or otherwise chemically active species.

Thermal bonding typically involves the introduction of heat between joined polymer layers by ultrasonic, linear or rotational agitation. Such physical agitation may often be damaging to active species, especially in the case of linear and rotational agitation where friction is generally used to broadly distribute heat across the bonding surface of a component. Solvents employed to prepare polymer surfaces for bonding may also be chemically destructive. Compression bonding typically requires temperatures on the order of the melting point of the substrate material, which often exceeds the temperatures at which attached biological agents, for example, remain active or otherwise useful. Additionally, the high pressure employed with compression bonding may result in mechanical shifting of the bonding layers thereby causing invasion into regions otherwise used for attachment of active species. The bonding of polymer layers may also be performed using various adhesives, such as tapes and epoxies; however, it may be possible for active species to adsorb to the adhesive material during manufacture and/or device use, thereby causing loss of function. Also, at the temperatures where endothermic reactions are typically observed, oligonucleotide hybridizations, enzyme functionality and/or cell lysis may destroy or otherwise compromise the integrity of an adhesive seal which may result in fluid leakage from the device or the release of chemical components from the adhesive that may interfere with the intended function of various active species.

Consequently, conventional methods of polymer bonding have generally not been regarded as suitable for integration with microfluidic technologies or in various applications requiring, for example, reduced form-factor, weight or other desired performance and/or fabrication process metrics. Moreover, previous attempts with the bonding of polymer layers in microfluidic devices have met with considerable difficulties in producing reliable fluidic connections and/or hermetic seals capable of withstanding manufacturing processes and/or operational stress while maintaining or otherwise reducing production costs. Accordingly, despite the efforts of the prior art to provide for the bonding of polymer layers in microfluidic systems, there remains a need for a low cost method for encapsulating and localizing active chemical species within the interior volume of a microfluidic device without damaging or otherwise functionally compromising the attached active species from operating for their intended purpose.

### SUMMARY OF THE INVENTION

In various representative aspects, the present invention provides a system and method for bonding polymer substrates in the presence of chemically active species. The process allows for substrate attachment of active species prior to bonding without subsequently damaging the active species or otherwise preventing them from properly functioning. A representative design is disclosed as comprising a thermoplastic mask sandwiched between two polymer substrate layers with at least one of the substrate layers having active species previously attached. The composite device may then be sealed with laser bonding where the wavelength λ of a laser may be selected so as not to damage the active species. In a representative aspect, the polymer substrates may be selected to demonstrate substantial transparency to the lasing wavelength λ while the thermoplastic mask may be selected to absorb radiation at the lasing wavelength λ in order inter alia to generate localized regions of heat to effectively bond the substrate layers to each other. The disclosed system and method, while well configured for producing devices for micro-assay applications, may be readily and more generally adapted for use in any microfluidic system. For example, the present invention may embody a device and/or method for use in micro-chromatographic separations, kinetics studies, spectroscopic characterizations and the like.

One representative advantage of the present invention would allow for improved process control and manufacturing of functionalized microfluidic channel surfaces at substantially lower cost. Additional advantages of the present invention will be set forth in the Detailed Description which follows and may be obvious from the Detailed Description or may be learned by practice of exemplary embodiments of the invention. Still other advantages of the invention may be realized by means of any of the instrumentalities, methods or combinations particularly pointed out in the claims.

### BRIEF DESCRIPTION OF THE DRAWINGS

Representative elements, operational features, applications and/or advantages of the present invention reside inter alia in the details of construction and operation as more fully hereafter depicted, described and claimed—reference being had to the accompanying drawings forming a part hereof, wherein like numerals refer to like parts throughout. Other elements, operational features, applications and/or advantages will become apparent to skilled artisans in light of certain exemplary embodiments recited in the detailed description, wherein:

FIG. 1 representatively depicts an elevation cross-section of an incoming substrate just prior to deposition and attachment of active species in accordance with an exemplary embodiment of the present invention;

FIG. 2 representatively illustrates deposition and attachment of active species on a surface of the substrate depicted, for example, in FIG. 1;

FIG. 3 representatively illustrates deposition of a radiation absorbing mask on the substrate depicted, for example, in FIG. 2;

FIG. 4 representatively illustrates deposition of a covering substrate over the active species and mask material depicted, for example, in FIG. 3;

FIG. 5 representatively illustrates radiative exposure of the mask material for the composite device depicted, for example, in FIG. 4; and

FIG. 6 depicts a elevation cross-section of the resulting sealed device representatively obtained from selective and localized heating of the mask material as illustrated, for example, in FIG. 5.

Those skilled in the art will appreciate that elements in the Figures are illustrated for simplicity and clarity and have not necessarily been drawn to scale. For example, the dimensions of some of the elements in the Figures may be exaggerated relative to other elements to help improve understanding of various embodiments of the present invention.

### DETAILED DESCRIPTION OF EXEMPLARY EMBODIMENTS

The following descriptions are of exemplary embodiments of the invention and the inventors' conceptions of the best mode and are not intended to limit the scope, applicability or configuration of the invention in any way. Rather, the following description is intended to provide convenient illustrations for implementing various embodiments of the invention. As will become apparent, changes may be made in the function and/or arrangement of any of the elements described in the disclosed exemplary embodiments without departing from the spirit and scope of the invention.

Various representative implementations of the present invention may be applied to any microfluidic system and/or method. As used herein, the terms “fluid”, “fluidic” and/or any contextual, variational or combinative referent thereof, are generally intended to include anything that may be regarded as at least being susceptible to characterization as generally referring to a gas, a liquid, a plasma and/or any matter, substance or combination of compounds substantially not in a solid or otherwise effectively immobile condensed phase. For example, a colloidal suspension may be considered to be a fluid inasmuch as colloids generally do not demonstrate the properties of an immobile condensed phase.

A detailed description of an exemplary application, namely a system and method for making functionalized microfluidic channel surfaces in a laminar device package is provided as a specific enabling disclosure that may be readily generalized by skilled artisans to any application of the disclosed system and method for providing microfluidic channels in accordance with various embodiments of the present invention. Moreover, skilled artisans will appreciate that the principles of the present invention may be employed to ascertain and/or realize any number of other benefits associated with providing functionalized microfluidic channels such as, but not limited to: reduction of device weight; reduction of device form factor; improved sample loading in microfluidic assays; improvement in sample throughput; sample multiplexing and/or parallel sample processing; integration with micro-array techniques and/or systems; microfluidic sample transport; as well as any other applications now known or hereafter developed or otherwise described in the art.

In an exemplary embodiment of the present invention, a system and method for the bonding of separate layers of polymeric substrates in the presence of attached chemically active species is disclosed. Various active species may be employed, such as, for example, elemental compounds, chemical compounds, molecules, biomolecules, proteins, amino acids, DNA, RNA, antibodies, antigens, enzymes, oligonucleotides and such other materials whether now known or otherwise hereafter described in the art.

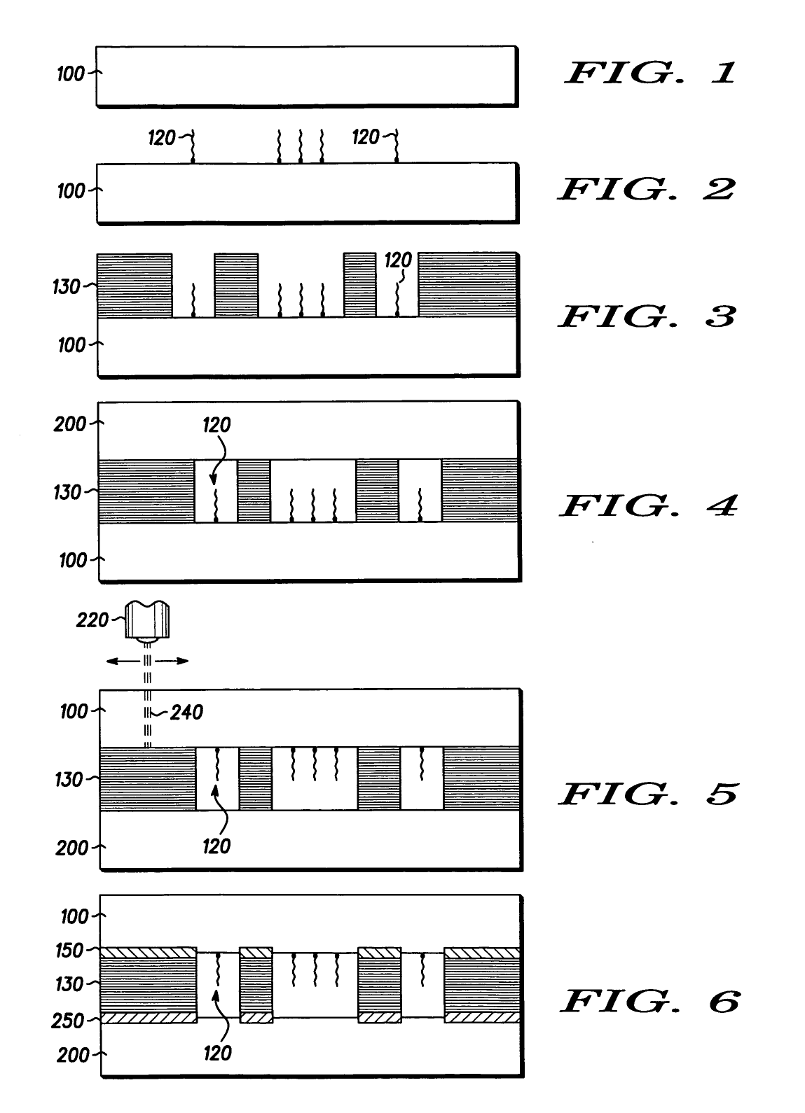

In one exemplary embodiment, incoming substrate 100 is oriented or otherwise suitably prepared to receive active species 120 for attachment to substrate 100, as depicted, for example, in FIG. 1. Incoming substrate 100 may comprise a polymer, glass, quartz, a mineral and/or any other material suitably adapted to the methods of fabrication herein disclosed, whether now known or otherwise hereafter described in the art. Thereafter, active species 120 are deposited and attached to substrate 100, as representatively illustrated, for example, in FIG. 2. A radiatively absorptive mask material 130 is then deposited over substrate 100 producing occluded volumes defining cavities comprising active species 120, as shown, for example, in FIG. 3. In an exemplary embodiment, mask material 130 comprises, for example, a thermoplastic compound and/or such other material or combination of materials whether now known or otherwise hereafter described in the art as capable of demonstrating the ability to absorb radiation of a predetermined wavelength. In a representative process, mask material 130 may be deposited via screen printing or such other methods or techniques whether now known, hereafter developed in the future or otherwise described in the art.

A second substrate 200 is then disposed over substrate 100 and mask 130 in order to cover the defined regions of active species attachment, as generally depicted, for example, in FIG. 4. Substrate 200 may comprise a polymer, glass, quartz, a mineral and/or any other material suitably adapted to the methods of fabrication herein disclosed, whether now known or otherwise hereafter described in the art. Thereafter, a radiative source 220 may be scanned over substrate surface 100 with radiation 240 of a user-selected wavelength in order to weld mask layer 130 to substrate layer 100, as representatively illustrated, for example, in FIG. 5. If the wavelength A of radiation 240 is (1) selected such that layer 100 is substantially transparent at that wavelength; (2) effectively non-destructive to active species 120; and (3) effectively absorbed by mask material 130, a welded bonding region 150 may be formed between substrate layer 100 and mask layer 130, as generally shown, for example, in FIG. 6.

In the case of mask layer 130 comprising a thermoplastic compound, absorption of radiation by the mask generally provides suitably adapted heating of the surfaces of substrates 100 and 200 that results in a robust welding of the surfaces. Accordingly, substrate layer 100 experiences only localized heating at the surface interface with, for example, thermoplastic layer 130. Utilizing radiation absorbing mask layer 130 in a substantially multi-layer format generally allows device features to be excised out of mask layer 130. As the radiation is scanned or otherwise exposed over the device layers during bonding, microfluidic channel and chamber features are generally defined by excised portions of the mask 130 typically not exposed to heat. Therefore, heat sensitive active species 120 attached to substrate layer 100 will generally not be proximally exposed to a heated surface and will therefore remain attached to the substrate surface 100 with preserved function for viable subsequent use.

Similar exposure of radiation 240 to the opposite side (e.g., through the surface normal to substrate layer 200) results in the formation of other bonding regions 250 between substrate layer 200 and mask layer 130, thereby effectively bonding substrate layer 100 to substrate layer 200 without effective damage to attached chemically active species 120.

Since wavelengths in the infrared range are relatively long with respect to visible and ultraviolet light and are not generally absorbed by biological or organic materials, in an exemplary embodiment of the present invention, radiative source 220 may comprise, for example, a collimated infrared lamp. In another representative application, radiation source 220 may be a laser. In yet another exemplary embodiment, radiation source 220 may comprise an infrared diode laser, wherein the diode laser may be optionally configured with a filter to produce substantially coherent monochromatic radiation of wavelength λ in the infrared region of the electromagnetic spectrum.

The method disclosed vida supra was tested in a microfluidic system by attachment of nucleic acid probes prior to channel sealing and assay hybridization. A serpentine channel feature was excised from a radiation-absorbing polycarbonate mask layer. 20 μM Escherichia Coli nucleic acid probes were attached to the surface of a radiation transmitting polycarbonate substrate layer in the areas corresponding to certain legs of the serpentine channel. 20 μM S. Sal nucleic acid probes were attached to the surface of a radiation transmitting polycarbonate substrate layer in the area corresponding to another channel leg to act as a negative control. The polycarbonate substrate layers were then laser bonded with an infrared diode laser. The bonding was repeated in order to fix a second polycarbonate substrate layer to cap the device thereby enclosing the microfluidic channels. After construction of the device was complete, a hybridization reaction was performed by adding 1 μM of complementary Escherichia Coli nucleic acid targets in 1×SSC for 1 hour at 50° C. The target nucleic acids were Cy3 fluorescently tagged. Signals were detected on a scanner at a wavelength of approximately 532 nm. The targets successfully hybridized to the attached complementary Escherichia Coli probes without observable hybridization of the negative control S. Sal probes. Additionally, the oligonucleotides present during the bonding process retained their structure and function.

In the foregoing specification, the invention has been described with reference to specific exemplary embodiments; however, it will be appreciated that various modifications and changes may be made without departing from the scope of the present invention as set forth in the claims below. The specification and figures are to be regarded in an illustrative manner, rather than a restrictive one and all such modifications are intended to be included within the scope of the present invention. Accordingly, the scope of the invention should be determined by the claims appended hereto and their legal equivalents rather than by merely the examples described above. For example, the steps recited in any method or process claims may be executed in any order and are not limited to the specific order presented in the claims. Additionally, the components and/or elements recited in any apparatus claims may be assembled or otherwise operationally configured in a variety of permutations to produce substantially the same result as the present invention and are accordingly not limited to the specific configuration recited in the claims.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components of any or all the claims.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted by those skilled in the art to specific environments, manufacturing specifications, design parameters or other operating requirements without departing from the general principles of the same.

1. A method for bonding substrate layers in the presence of chemically active species to form a functionalized microfluidic surface, said method comprising the steps of:
providing a first substrate;
depositing an active species on said first substrate;
depositing a radiatively absorptive mask on said first substrate;
disposing a second substrate over said radiatively absorptive mask; and
exposing said mask to radiation so as to effectively positionally fix said first substrate with respect to said second substrate to produce a microfluidic channel.

2. The method of claim 1, wherein at least one of said first substrate and said second substrate comprise at least one of a polymer, glass, quartz and a mineral.

3. The method of claim 1, wherein said active species comprises at least one of an elemental compound, a molecule, a biomolecule, a protein, an amino acid, DNA, RNA, an antibody, an antigen and an enzyme.

4. The method of claim 1, wherein said mask comprises a thermoplastic compound.

5. The method of claim 1, wherein said deposition of said mask further comprises the step of screen printing.

6. The method of claim 1, wherein said radiation comprises at least one of electromagnetic radiation, microwave radiation, radio frequency radiation and infrared radiation.

7. The method of claim 6, wherein the wavelength of said radiation is substantially monochromatic.

8. The method of claim 7, wherein said wavelength of said radiation is selected to demonstrate effective transmission in at least one of said first substrate and said second substrate.

9. The method of claim 7, wherein said wavelength of said radiation is selected to demonstrate effective absorption in said mask.

10. The method of claim 1, wherein the material of at least one of said first substrate and said second substrate is selected to demonstrate effective transparency to said radiation.

11. The method of claim 1, wherein said mask is selected to demonstrate effective absorption of said radiation.

12. A microfluidic device fabricated in accordance with the method of claim 1.

13. The microfluidic device of claim 12, wherein at least one of said first substrate and said second substrate comprise at least one of a polymer, glass, quartz and a mineral.

14. The microfluidic device of claim 12, wherein said chemically active species comprises at least one of an elemental compound, a molecule, a biomolecule, a protein, an amino acid, DNA, RNA, an antibody, an antigen and an enzyme.

15. The microfluidic device of claim 12, wherein said radiatively absorptive mask comprises a thermoplastic compound.

16. The microfluidic device of claim 12, wherein the material of at least one of said first substrate and said second substrate is selected to demonstrate effective transparency to electromagnetic radiation of a predetermined wavelength.

17. The microfluidic device of claim 12, wherein said radiatively absorptive mask material is selected to demonstrate effective absorption of electromagnetic radiation of a predetermined wavelength.

18. A method for bonding substrate layers in the presence of oligonucleotide probe species to form a functionalized microfluidic channel surface, comprising the steps of:
providing a first polycarbonate substrate;
depositing said oligonucleotides on said first substrate;
depositing a thermoplastic mask on said first substrate;
disposing a second polycarbonate substrate over said thermoplastic mask; and
exposing said mask to radiation so as to effectively bond said second substrate with said first substrate to produce a microfluidic channel.

19. The method of claim 18, wherein the source of said radiation comprises an infrared diode laser.

20. The method of claim 18, further comprising the step of detecting an analyte signal by interrogation of said probe species.

21. The method of claim 20, wherein said detection mechanism comprises measurement of a fluorescence intensity.
